(19) United States
(12) Patent Application Publication
(10) Pub. No.: US 2006/0217690 A1
(43) Pub. Date: Sep. 28, 2006

(54) Method for treating various dermatological and muscular conditions using electromagnetic radiation

(76) Inventors: Norman J. Bastin, Encinitas, CA (US); Stanley D. Frank, Delray Beach, FL (US); Dan Watts, North Aurora, IL (US)

Correspondence Address: FISH & RICHARDSON, PC, P.O. BOX 1022, MINNEAPOLIS, MN, 55440-1022, US

(21) Appl. No.: 11/087,381

(22) Filed: Mar. 22, 2005

(51) Int. Cl. A61B 18/18

(52) U.S. Cl. 606/9; 607/88

(57) ABSTRACT

Provided are methods and devices for light therapy. The devices and methods are useful for the treatment of a number of skin tissue conditions and disorders. The device includes an array of light emitting devices on a flexible, formable sheet useful for exposing a plurality of surfaces to photomodulation. The light is substantially monochromatic.

### TECHNICAL FIELD

The invention generally relates to a device and method for treating skin or hair conditions and disorders with light therapy alone or in combination topical treatment compositions.

### BACKGROUND

Light therapy is an emerging field that utilizes low non-thermal electromagnetic radiation to treat skin and tissue disorders including acne, hair growth stimulation, hair growth inhibition, scar reduction and removal, wrinkle reduction, and the like. Current low non-thermal electromagnetic radiation treatment tends to be a broad range of between 20-690 nm or more and is not specific for any particular condition or disorder.

### SUMMARY

This document discloses a system and method for treating skin using photo light therapy. In one aspect, a method for treating a skin condition or disorder includes the steps of pretreating a skin tissue by contacting the skin tissue with a phyto-enriched composition, and contacting the skin tissue with a microabrasion composition. The method further includes exposing a skin tissue to at least one specific peak band wavelength with a range of ±10 nm that is specific for the condition or disorder to be treated.

In another aspect, a device includes a formable, flexible material comprising a first surface and a second surface, and a plurality of light emitting devices on the first surface of the formable, flexible material. The device further includes an electronic driving component for driving the plurality of light emitting devices, disposed on the second surface. The device further includes a control component that controls the pulse and intensity of the light emitting devices.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1A-D shows a device of the invention. (A) shows a light emitting device array comprising a formable, flexible surface having a plurality of light emitting diodes thereon; (B) is a diagram showing the use of a device of the invention on the face; (C) shows the use of the device on the scalp; and (D) shows the use of the device on the chest.

FIG. 2A-C show another aspect of a device of the invention. (A) shows a hand and foot light device comprising an array of lights; (B) shows a schematic of the device of (A) with wells on a surface of the device; and (C) shows is a diagram showing the use of the device on the hands of a subject.

FIG. 3A-B show actual photos of a device of the invention.

FIG. 4A-D are schematics showing the electronics of an array of lights for use with the invention.

Like reference symbols in the various drawings indicate like elements.

### DETAILED DESCRIPTION

This document discloses methods and devices for skin and hair tissue treatment (e.g., dermatologic treatment). These methods and devices are useful for the treatment of a number of skin disorders including hair loss, hair growth, acne, scarring, cellulite, fine lines and wrinkles. These methods and devices utilizes narrow band, substantially monochromatic light for the treatment of such conditions and disorders. The methods and devices can be powered by alternating current or direct current (i.e. battery).

A narrow band, substantially monochromatic light comprises a light source that has a single peak wavelength with minimal bandwidth. In one aspect, light consist of a single wavelength. In another aspect, the light comprises a peak wavelength having a bandwidth of ±2 nm, ±3 nm, ±4 nm, ±5 nm, ±6 nm, ±7 nm, ±8 nm, ±9 nm, or ±10 nm. The narrowband width provides optimal light energy designed to treat a specific condition, or disorder that is receptive to that wavelength. The reduced bandwidth compared to prior light therapy methods and devices reduces the thermal energy applied to a skin surface thereby providing a more effective treatment compared to the prior methods and devices.

The light used in the methods and devices disclosed herein can be obtained/produced by a light emitting diode (LED), an organic light emitting diode (OLEDs), a sulfur lamp, an arc lamp (e.g., a xenon lamp), a light emitting polymer, a metal halide lamp, a filamentous light source, and a laser light source. Any of the above light sources may be used in conjunction with a filter that filters out undesirable wavelengths and/or to produce a bandwidth of 20 nm or less from the peak bandwidth.

Although the use of LEDs is described, the invention is not limited solely to the use of LED sources. For example, any light source capable of emitting electromagnetic radiation that meets a substantially monochromatic wavelength (e.g., ±10 nm), as described herein, directly, or by means of optical, electronic, or mechanical filtration, is a suitable light source for use in the methods and compositions of the invention.

Where LED are not used and where a light is filtered, density filters are particularly useful for modifying the emission of light to deliver a non-thermally damaging and safe amount to a skin tissue. The targeted skin or tissue may be exposed to one or more peak, non-overlapping wavelengths of light. The methods and devices disclosed herein are configured to expose a skin tissue to particular wavelengths of light or a combination of certain wavelengths of light.

One of skill in the art will recognize that light energy will cause some slight increase in skin tissue temperature. The light energy provided by the methods and devices of the invention will not provide energy to cause thermal injury or adverse effects related to thermal energy. The methods and devices disclosed herein include systems and techniques that modify the duration and/or frequency of light contacting the skin to maintain a relatively even temperature at the skin surface or a desired temperature at the skin surface. For example, a pulse of between 1 femtoseconds to 5000 femtoseconds may be effective, although greater or lesser pulse periods may also be used.

The methods and devices disclosed herein use narrow bandwidth, substantially monochromatic light to treat skin conditions and disorders. By eliminating overly broad bandwidth (e.g., greater than 20 nm), the invention reduces unwanted light effects (e.g., thermal effects). For example, if a subject is being treated for acne, specific bands of light are used for treatment. If the wavelength band is overly broad to encompass wavelengths that induce hair growth or hair loss, the result will be reduced acne with unwanted side effects (e.g., hair growth or hair loss). Thus, if the light bandwidth is too broad, the desired effects may be altered from those intended. Accordingly, use of broad band (e.g., greater than 20 nm) light sources are less desirable. For purposes of the invention, any device that emits light in a bandwidth of ±about 10 nanometers or less around a dominant wavelength is a narrowband, substantially monochromatic light source. Most commercial lasers emit light at a single wavelength of light and are considered monochromatic.

Light sources can be used in either a continuous or in a pulsed short manner. A LED or non-laser light source could produce a continuous or pseudo-continuous beam of light energy using pulse durations. A pulse duration of between about 0-2000 pulses per second is typically used.

Depending upon the wavelength, different tissues and penetrations may be achieved. In addition, skin tissues and penetration will be different for different age groups, conditions including diseases or disorders, skin tone, color, and ethnicity. Furthermore, skin penetration can be modified by treating the skin tissue prior to exposure. For example, skin that has been abraded or peeled will have a different penetration than non-treated skin tissue. For example, light having a peak wavelength emission of about 514 nm ±10 nm is sufficient to penetrate all sebaceous glands or acne cysts but by itself may not be effective to clear overall skin disorders due to the limited depth of penetration. In contrast, light having a wavelength of about 650 nm ±10 nm can more easily penetrate to a greater depth. Thus, a combination therapy may be more effective in treating a particular condition, such as acne, for example. As discussed more fully elsewhere herein, the addition of topical compositions comprising agents that absorb electromagnetic radiation will also effect the absorbance and penetration of particular wavelengths of light.

The light source used may be provided in a array of individual light emitters such as a plurality of LEDs in a formable/flexible sheet. Such a formable sheet can be used to adjust or flex to the contours of the skin tissue being exposed to light. For example, a formable sheet of LEDs can be formed into the shape of a mask that can be applied to the face or to the shape of the chest, neck, back, arm, legs, groin, buttocks, and the like. LED sources are considered insignificant risk devices, thus minimal supervision is required. Such devices can be adapted for home use. Accordingly, the array can comprise a formable sheet that can be manipulated to meet the curvature of the body area to be treated.

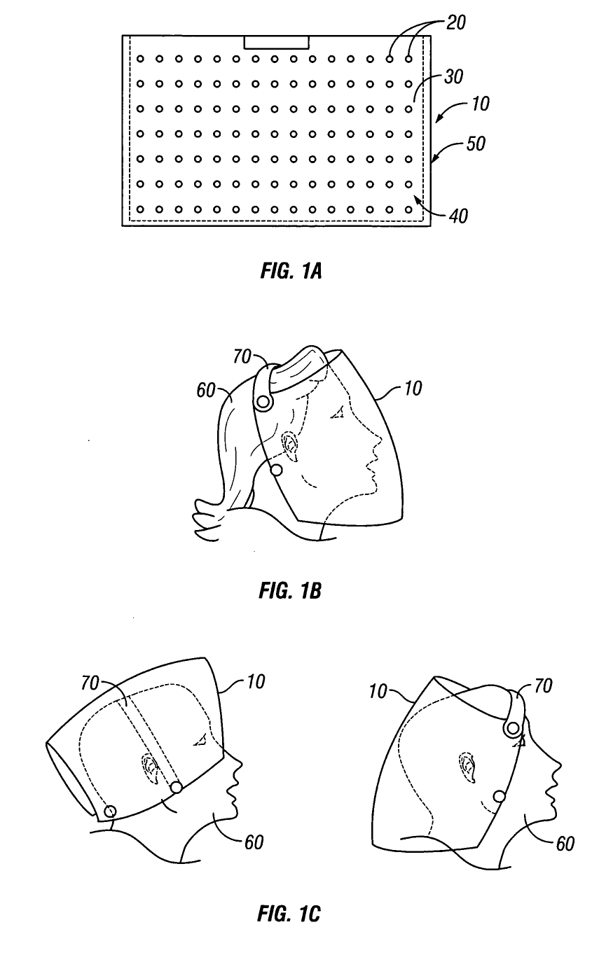

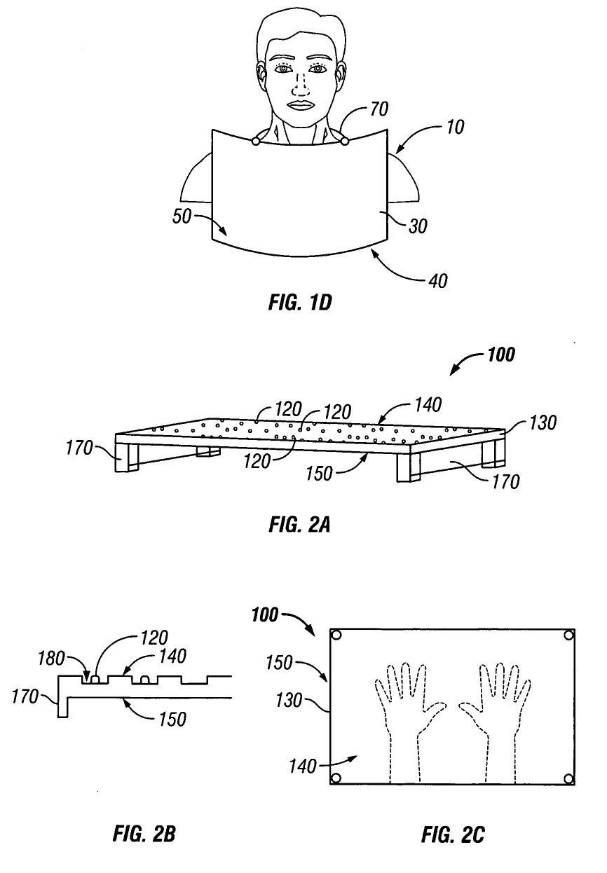

Referring now to FIG. 1A there is depicted an LED array 10 comprising a plurality of LEDS 20. The LEDs can be from any commercial source. The LEDs are typically uniformly spaced and attached to a formable/flexible material 30. The formable material 30 can comprise a fabric, a polymer, a foam, a metal and the like. In one aspect, the LEDs are mounted on a material commonly known as “G10”. The G10 material is an epoxy impregnated type of fiberglass. For example, G10 circuit board material that is about 0.010″ to about 0.020″ thick, e.g., 0.015″ (although other thickness is available from about 0.002″ to several inches) is sufficiently flexible. The formable material 30 comprises and inside surface 40 and outside surface 50. The LEDs are positioned such that the light emitted from the LEDs contacts a skin surface that is 10-25 mn from the inside surface 40. Typically the electronics for powering and controlling the LEDs are present on the outside surface 50.

FIG. 1B shows the use of LED array 10 on the face of a subject 60. In this aspect, the formable material 30 is wrapped round the subject's face and held in place by one or more fastening straps 70 or spring loaded formable matter to attach to the area behind the subject's ears. FIG. 1C shows the use of an LED array 10 to treat the skin tissue of the scalp. FIG. 1D shows use of the LED array 10 on the chest of the subject. In this embodiment, the LEDs 20 are exposed to the skin tissue of the chest. A fastening strap 70 another means around the subject's neck holds the LED array 10 in place during use.

The lights can be configured or controlled to dim or flash at predetermined time intervals to indicate a remaining time for a particular treatment or session. For example, the lights can be configured to flash three times to indicate that 15 minutes remain in a session, flash two times to indicate 10 minutes remaining, and flash one time to indicate a remaining time of 5 minutes. Various other combinations of flashing and/or dimming can be used. The brightness of the lights can be controlled to increase slowly when a cycle or session is first started, or when LEDs are turned off to allow a user's eyes time to adjust to the outside light.

The light pattern is configurable in various ways. In one embodiment, one column of LEDs is made to be brighter than other columns, and the bright column can be controlled to move across the array of columns in a back-and-forth manner. In another embodiment, the LEDs are dimmed at the end of a cycle, or when the session is paused. Each photo light therapy treatment session can be preset for a particular time or interval.

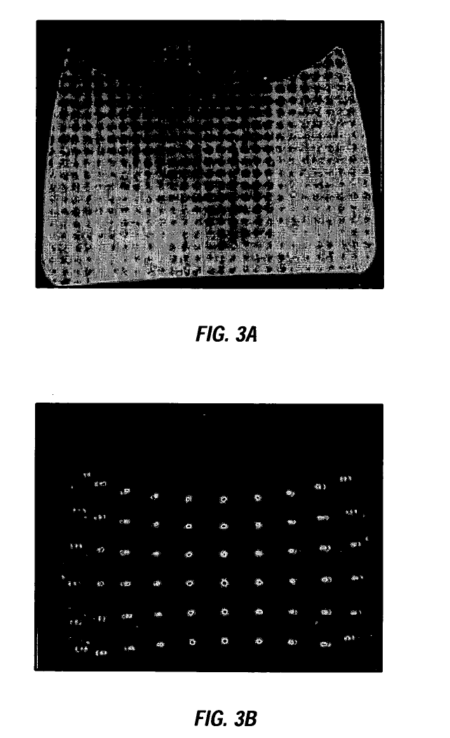

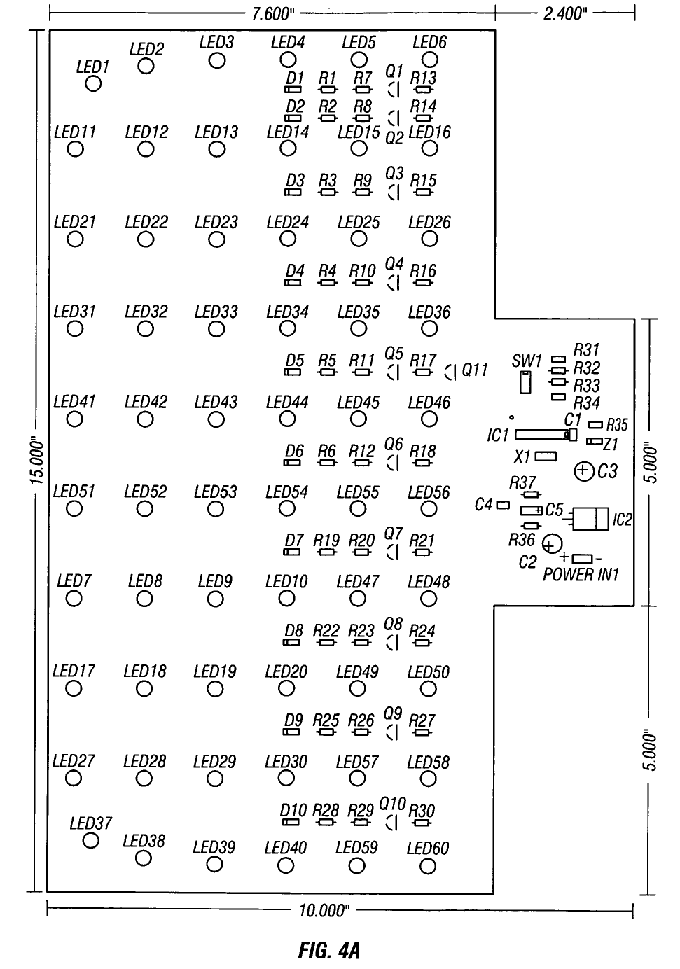

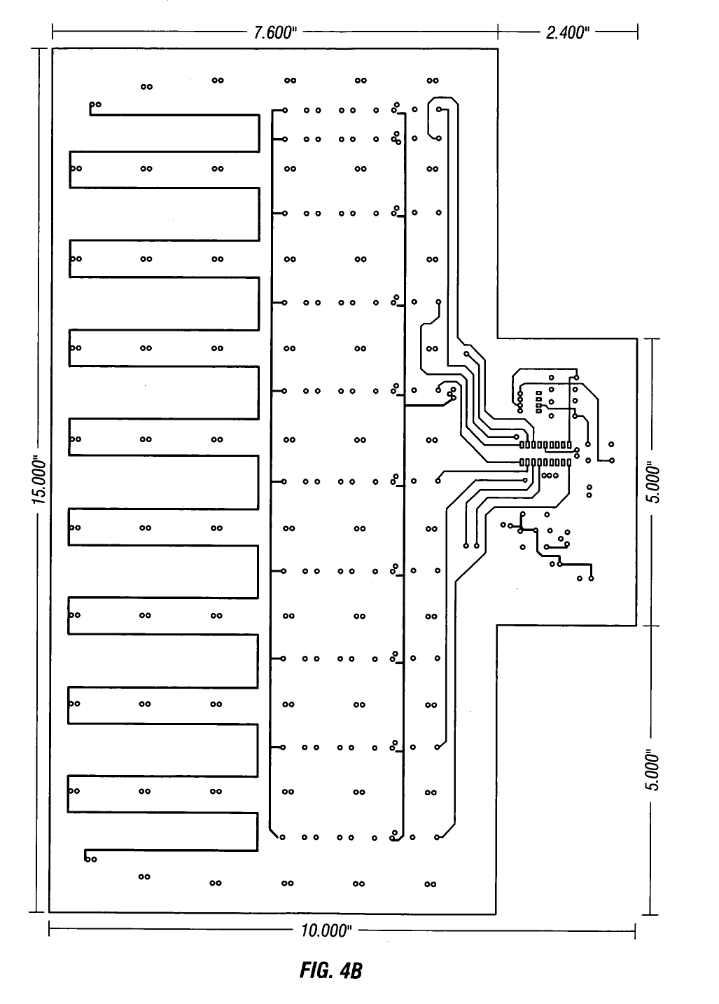

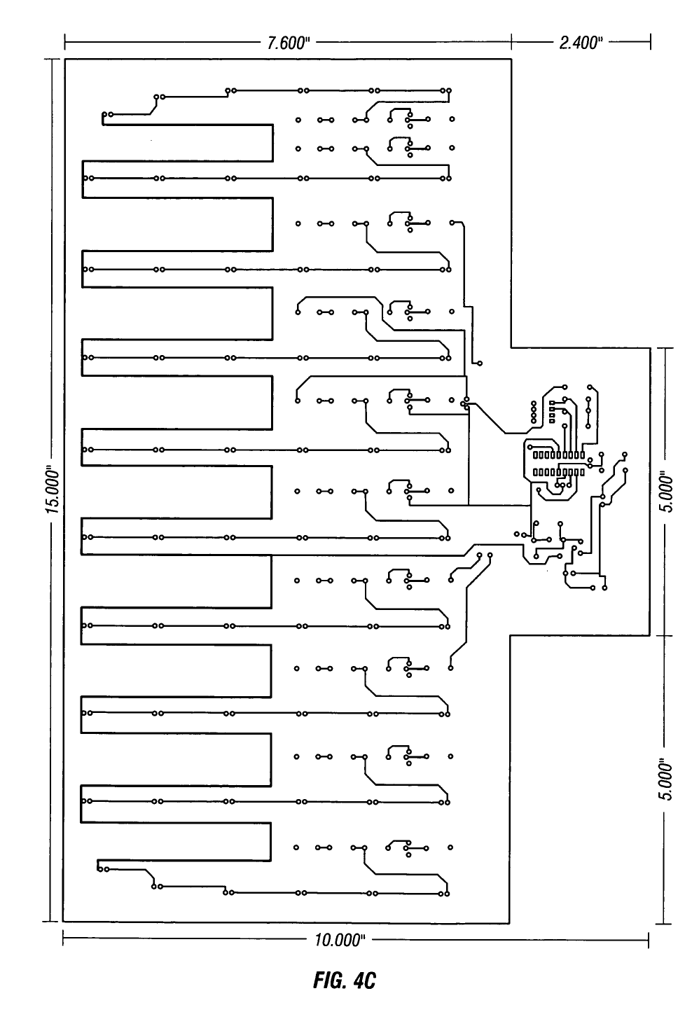

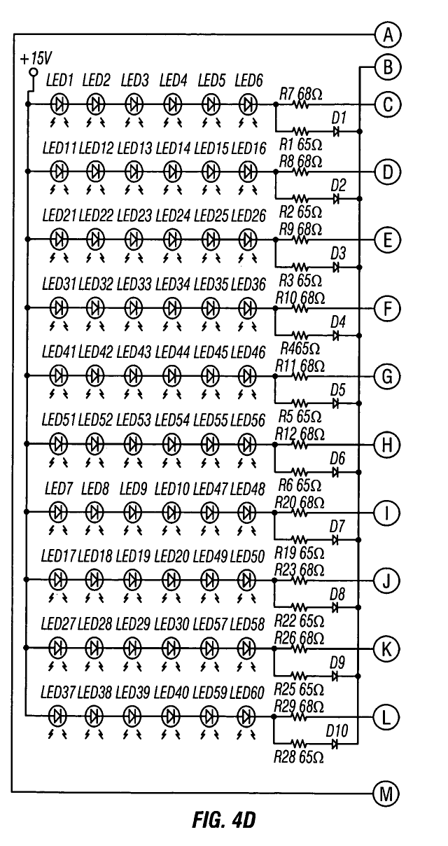

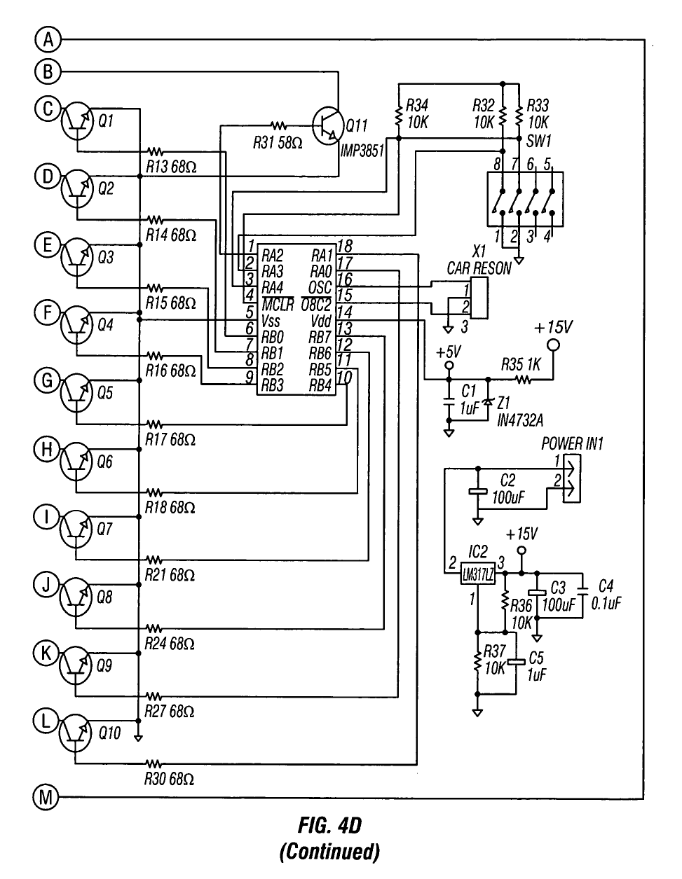

In a further aspect and array of LEDs may be provided on a flat surface either attached to formable material or on a rigid material. FIG. 2A shows a flat panel system 100. The flat panel system comprises a top surface 140 and bottom surface 150. A plurality of LEDs 120 can be disposed such on surface 140 or on surface 150 or wherein each LED is disposed in a well 180 (see FIG. 2B). Each well may include reflective material. The panel may include legs 170. In one aspect, the legs serve to lift the surface platform 130 to allow a subject to slide their hands or feet under the platform 130 where the LEDs are disposed on surface 150. In another aspect, the electronics are disposed on the surface opposite of the LEDs. In a further aspect, the platform is sufficiently sturdy to stand on such that the skin tissue of the top and/or bottom of the feet is exposed to light from the LEDS to. FIG. 2C shows the use of array 100 with a subject's hands. One or both hands may be placed on or under the platform 130. FIG. 3A-B show actual photos of a device of the invention. FIG. 4A-D are schematics showing the electronics of an array of lights for use with the device.

Two entirely different light wavelengths may be used simultaneously or sequentially in the methods and devices disclosed herein. For example, additive therapeutic effects may be achieved by using two wavelengths at the same time such as for the treatment of acne. The use of blue light with a wavelength of approximately 514 nm and red light with a wavelength of approximately 650 nm.

In one embodiment, topical compositions are used in connection with the light therapy. The topical compositions include a pretreatment composition useful to rid the skin of oils and atmospheric particles that may clog the skin's pores. The pretreatment/prewash compositions is a phyto-enriched compositions that prepares the skin for dermabrasion treatment and light therapy. In one aspect, the pretreatment/prewash compositions comprises steam-distilled water, aloe vera, decyl glucoside, copper-containing peptides, a 5% citric acid mix, green tea extract, xanthan gum, and citric oils (e.g., lime oil, lemon oil grapefruit oil, tangerine oil and lemongrass oil).

In another aspect, a dermabrasion topical treatment is used prior to light therapy. The dermabrasion topical treatment can be used alone or in combination with the pretreatment/prewash composition above. A dermabrasion topical composition comprises pharmaceutical grade, non-dissipating microdermabrasion creme for skin reddening and resurfacing. A number of dermabrasion compositions are available on the market. In one aspect, the dermabrasion composition comprises steam-distilled water, aluminum oxide, sunflower oil, cetearyl glucoside, glycerol stearate, cetearth alcohol, palmitoyl-pentapeptide, vegetable glycerine, grapeseed oil, almond oil, caprylic acid glycine xanathan gum, potassium sorbate, jojoba oil, vitamin E, Lecithin, methylparaben, and propylparaben. A further topical treatment can be used to optimize light therapy including the use of light absorbing agents and the like.

Other agents that can be combined with either or both of the foregoing compositions include, e.g., Vitamin C, Vitamin E, Vitamin D, Vitamin A, Vitamin K, Vitamin F, Retin A (Tretinoin), Adapalene, Retinol, Hydroquinone, Kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, algae, an antioxidant, a phytoanthocyanin, a phytonutrient, plankton, a botanical product, a herbaceous product, a hormone, an enzyme, a mineral, a genetically engineered substance, a cofactor, a catalyst, an antiaging substance, insulin, trace elements (including ionic calcium, magnesium, etc), minerals, minoxidil, a natural or synthetic melanin, a metalloproteinase inhibitor, proline, hydroxyproline, an anesthetic substance, chlorophyll, bacteriochlorophyll, copper chlorophyllin, chloroplasts, carotenoids, phycobilin, rhodopsin, anthocyanin, and combinations thereof.

Following light therapy an additional topical composition may be applied to the treated skin tissue. This post-treatment composition is useful to stimulate the production of skin quenching repair fluids to nourish and feed the increased production of collagen. In one aspect, the composition comprises steam-distilled water, herbal infused green tea extract, idebenone, grapsee extract, cyclomethicone and dimethicone crosspolymer, vitamin C ester, alpha lipoic acid, cetearyl glucoside, potassium sorbate, DMEA bitartate, vegetable glycine, squalene, NMF, hyalurnic acid, ubiquinol CQ10, panthenol, allontoin, soy phospholipids liposome gel, whole wheat protein, vitamin A, methylparaben, and propylparaben. In certain aspect, the post-treatment composition comprises an agent the modulates the activity of photolyase.

In accordance with one exemplary embodiment, a method of treating acne is provided. Acne is the result of a combination of biological factors that ultimately result in blockage and buildup in the sebaceous glands. Propionibacterium acnes is a bacteria present in the sebaceous glands that is a common cause of acne. The presence of buildup and/or bacteria in the sebaceous glands causes an inflammatory reaction leading to pustules on the skin. Acne may be exacerbated by iodides, and exposure to hydrocarbons or bromides. In one embodiment, a process may be used to provide short or long-term control, improvement, reduction or elimination of acne or other related skin conditions. The process utilizes a combination of non-overlapping peak light wavelengths to treat acne conditions and disorders. The light waves comprise a first narrow bandwidth substantially monochromatic light of about 514 nm ±10 nm and a second narrow bandwidth, substantially monochromatic light of about 650 nm ±10 nm.

In a further aspect of treating acne, an agent may be physically or chemically or immunologically incorporated into the sebaceous (oil) glands, ducts, or supporting tissue, or into the naturally occurring acne bacteria prior to treatment. Other similar disorders such as folliculitis which involve the pilosebaceous (hair/oil gland) unit may also be treated using the invention. The invention may also be used to reduce perspiration, sweating, or hyperhidrosis from eccrine (sweat) glands or apocrine glands. The methods and devices disclosed herein may be used to treat other skin disorders such as, for example, warts, psoriasis, precancerous skin lesions, and diabetic, pressure, venous stasis skin ulcers.

In addition, the light therapy methods and devices are useful for reducing and eliminating common acne bacteria. For example, the light therapy techniques and devices can be used to reduce or eliminate the presence of acnes vulgaris and for safely treating conditions such as pseudofolliculitis barbae, acne rosacea, and sebaceous hyperplasia. Antibiotics that kill bacteria present in the sebaceous glands can be used in combination with the light therapy described herein.

In one embodiment, light absorbing molecules that absorb light in the 500-520 nm range and/or the 640 to 66 nm range or contacted with the skin tissue prior to treating an acne disorder. For example, chlorophyll or its derivatives or other related plant or dye light absorbing agents are contacted with the skin tissue such that the are absorbed and/or located to the sebaceous gland and surrounding tissue. The skin tissue comprising the light adsorbing molecule(s) is then exposed to LED, or other light sources that provides a wavelength of 514 nm ±10 nm and/or 640 nm ±10 nm. Light exposure to the skin tissue is provided at a sufficient energy and time (e.g., pulse duration) to allow to sufficiently inhibit or reduce acne bacteria content and to reduce or destroy gland activity.

Chlorophyll A, for example, exhibits an absorption maxima at 409 nm very close to one of the peak wavelengths (e.g., 410 nm) used in the methods of the invention. Chlorophyll B exhibits an absorption maxima at 642 nm, very close to one of the peak wavelengths (e.g., 640 nm) used in the methods of the invention. It can be readily seen that various types of chlorophyll, or combinations thereof, can be used as topically applied light absorbing agents to assist the absorption of certain wavelengths of light delivered to the skin tissue for various treatments (e.g., acne treatment).

In one aspect, a combination of topical formulations and light therapy is provided to the skin tissue comprising acne to produce a desired frequency level to destroy bacteria and inhibit sebaceous gland oil production. For example, the presence of a light absorbing molecule in the sebaceous gland can absorb light energy to increase the temperature in the gland thereby killing the bacterial cells and sebaceous gland oil producing cells.

In accordance with an exemplary embodiment, a method and device includes the use of two or more wavelengths that can be applied to a skin tissue. In one aspect, the methods and devices can provide two different light sources or wavelengths sequentially or simultaneous to have different effects such as treating active acne lesions and also acne scarring; treating acne rosacea lesions and also rosacea blood vessels or telangectasia. As described herein, the method of treating acne utilizes to non-overlapping peak wavelengths of 514 nm ±10 nm and 650 nm ±10 nm. These two wavelengths may be provided simultaneously or sequentially to the skin tissue to be treated.

As described above, an array of LEDs can be used in the light therapy methods for treating acne. In this aspect, the LED array is positioned over the skin tissue to be treated. For example, the light array can be applied to the face as in FIG. 1B or to the chest or back as in FIG. 1D to treat acne of the face, chest or back, respectively.

The methods and devices can also be applied for reducing cellulite, using light therapy to improve circulation at the site of cellulite. Although not wishing to be bound by any theory, it is believed that the light therapy methods of the invention improve circulation by inducing vasodilation of the skin tissue. In order to cause sufficient vasodilation, the penetration of the light into the skin tissue should be sufficient to not only induce surface vasodilation, but vasodilation of underlying tissue. Accordingly, longer wavelengths of light are typically used in such methods. For example, using an LED array of the invention, light is provided to the skin tissue to be treated in a wavelength of about 650 nm ±10 nm. To enhance treatment, and penetration the skin tissue may be pretreated and topically treated as described herein. In one aspect, a topical application of a light absorbing molecule may be applied that absorbs light in the 640 to 660 nm range thereby allowing for sufficient photo energy introduced to the skin cells.

The invention also provides methods of increasing hair growth by contacting a skin tissue (e.g., the scalp) with light sufficient to improve blood flow to the tissue and with the production of protein by the hair follicles. In order to cause sufficient vasodilation, the penetration of the light into the skin tissue should be sufficient to not only induce surface vasodilation, but changes in the underlying tissue to produce protein. Accordingly, longer wavelengths of light are typically used in methods to stimulate hair growth. For example, using an LED array of the invention, light is provided to the skin tissue to be treated in a wavelength of about 650 nm ±10 nm. To enhance treatment, and penetration the skin tissue may be pretreated and topically treated as described herein. In one aspect, a topical application of a light absorbing substance may be applied that increased the absorption of light in the 640 to 660 nm range thereby allowing for increased photo energy of the skin/scalp.

As discussed herein, the skin tissue may be treated to improve permeability of the light and/or topical agents. This may be accomplished, for example, by pretreating the skin with the pretreatment compositions disclosed herein to hydrate the skin and/or remove the stratum corneum, dirt, debris, oils, and the like. In one aspect the scalp is pretreated and then a dermabrasion composition is used to remove the stratum corneum to promote light penetration and topical treatment with light absorbing agents and/or therapeutic agents.

In other aspects, where the composition comprises light absorbing substance, the light absorbing substances are typically naturally occurring non-toxic, biocompatible agents. Such agents include, for example, chromophore such as chlorophyll, chlorophyllin, protoporphyin, bacteriochlorophyll, and the like. Such agents may be delivered in pure form, in solution, in suspension, in emulsions, in liposomes, in synthetic or natural microspheres, microsponges or other known microencapsulation or non encapsulation vehicles, alone or in combination.

Scarring is sometimes seen as a consequence of skin conditions, injury and disorders. Scarring may consist of one or more of the following: raised hypertrophic scars or fibrosis, depressed atrophic scars, hyperpigmentation, hyperpigmentary redness or telangectasia. Raised or thick or hard hypertrophic scars. Light stimulation of scar skin tissue has been shown to induce the production and/or activity of proteases in the skin (e.g., matrix metalloproteases). These proteases are typically active in fresh wounds and play a role in dissolving dead or dying tissue to promote clean healing of the wound and also serve to destroy invading germs (e.g., bacteria and viruses). The methods and devices of the invention can be used to stimulate the production of collagen dissolving proteases resulting in reduced scar tissue.

As described herein, the invention provides use of light therapy alone or in conjunction with a topical light absorbing agent, typically after skin treatment comprising dermabrasion. In a further aspect, the invention also provides concomitant use of an oral or systemically delivered agent. The oral composition comprises one or more of the following agents: APC, vitamin A, vitamin C, vitamin D3, vitamin E, Niacin, vitamin B6, folic acid, vitamin B12, PABA, grape extract, sulfur containing compounds (e.g., MSM etc.), alpha lipoic acid, flax seed oil, L-tyrosine, L-valine, and glucosamine sulfate.

Exogenous light absorbing molecules include agents that absorb light or in at least one narrow band of wavelengths and assist. The selection of the exogenous light absorbing molecules is determined by the wavelength of the narrowband substantially monochromatic light used for treatment. In some aspect, the light absorbing molecules will aid in treatment by adsorbing a desired wavelength of light at a particular depth or location (i.e., the depth of penetration or location of the agent) to photo treat or photo modify bacteria, tissue, glands, ducts and the like.

The invention may be used with or without the application of a topical composition to the skin tissue. One function of such compositions is to allow penetration of the light waves or to modify the refractive index of the skin tissue.

A number of embodiments of the invention have been described. Nevertheless, it will be understood that various modifications may be made without departing from the spirit and scope of the invention. Accordingly, other embodiments are within the scope of the following claims.

What is claimed is:

1. A method for treating a skin condition or disorder, comprising:
pretreating a skin tissue by contacting the skin tissue with a phyto-enriched composition;
contacting the skin tissue with a microabrasion composition; and
exposing a skin tissue to at least one specific peak band wavelength with a range of ±10 nm that is specific for the condition or disorder to be treated.

2. A method in accordance with claim 1, wherein the condition or disorder is selected from the group consisting of hair loss, hair growth, wrinkles, wound, scarring, and acne.

3. A method in accordance with claim 1, wherein the condition or disorder is acne.

4. A method in accordance with claim 3, wherein the at least one specific peak band wavelength is 650 nm ±10 nm.

5. A method in accordance with claim 3, wherein the at least one specific peak band wavelength is 514 nm ±10 nm.

6. A method in accordance with claim 3, wherein the at least one specific peak band wavelength comprises two non-overlapping wavelengths.

7. A method in accordance with claim 6, wherein the non-overlapping wavelengths comprise 650 nm ±10 nm and 514 nm ±10 nm.

8. A method in accordance with claim 1, wherein the condition or disorder is hair loss and thickening.

9. A method in accordance with claim 8, wherein the at least one specific peak band wavelength is 650 nm ±10 nm.

10. A method in accordance with claim 1, wherein the condition or disorder is would healing scarring or wrinkles.

11. A method in accordance with claim 10, wherein the at least one specific peak band wavelength is 650 ±10 nm.

12. A method in accordance with claim 1, wherein the at least one specific peak band wavelength is pulsed on and off from 1 to 2000 times per second.

13. A method in accordance with claim 7, wherein the non-overlapping wavelengths are alternatively cycled during exposure.

14. A method in accordance with claim 7, wherein the non-overlapping wavelengths simultaneously exposed to the skin tissue.

15. A method in accordance with claim 1, wherein the at least one specific peak band wavelength exposes the skin from 0 joules/cm² to about 4.2 joules/cm², ±0.2 joules/cm².

16. A method in accordance with claim 1, further comprising contacting the skin tissue with a topical composition comprising a chromophore prior to exposing the skin tissue to the at least one specific peak band wavelength.

17. A method in accordance with claim 1, further comprising administering a nutraceutical composition to a subject prior to contacting the subject's skin tissue with the at least one specific peak band wavelength.

18. A method in accordance with claim 1, wherein the at least one specific peak band wavelength is provided by a light source selected from the group consisting of a light, emitting diode, a fluorescent light source, an organic light emitting diode, a light emitting polymer, a xenon arc lamp, a metal halide lamp, a filamentous light source, an intense pulsed light source, a sulfur lamp, and combinations thereof.

19. A method in accordance with claim 16, wherein the topical composition is selected from the group consisting of naturally occurring chlorophyll-containing compounds, carotenoid-containing compounds, phyocobilin compounds, indocyanine green, methylene blue, rose Bengal, Vitamin C, Vitamin E, Vitamin D, Vitamin A, Vitamin K, Vitamin F, Retin A (Tretinoin), Adapalene, Retinol, Hydroquinone, Kojic acid, a growth factor, echinacea, an antibiotic, an antifungal, an antiviral, a bleaching agent, an alpha hydroxy acid, a beta hydroxy acid, salicylic acid, antioxidant triad compound, a seaweed derivative, a salt water derivative, algae, an antioxidant, a phytoanthocyanin, a phytonutrient, plankton, a botanical product, a herbaceous product, a hormone, an enzyme, a mineral, a cofactor, an antiaging substance, insulin, minoxidil, lycopene, a natural or synthetic melanin, a metalloproteinase inhibitor, proline, hydroxyproline, an anesthetic, chlorophyll, bacteriochlorophyll, copper chlorophyllin, chloroplasts, carotenoids, phycobilin, rhodopsin, anthocyanin, inhibitors of ornithine decarboxylase, inhibitors of vascular endothelial growth factor (VEGF), inhibitors of phospholipase A2, inhibitors of S-adenosylmethionine, licorice, licochalone A, genestein, soy isoflavones, phtyoestrogens, derivative, analogs, homologs, and subcomponents thereof, and derivatives, subcomponents, immunological complexes and antibodies of said target tissue, and synthetic and natural analogs thereof, and combinations thereof.

20. A device comprising
a formable, flexible material comprising a first surface and a second surface;
a plurality of light emitting devices on the first surface of the formable, flexible material;
an electronic driving component for driving the plurality of light emitting devices, disposed on the second surface; and
a control component that controls the pulse and intensity of the light emitting devices.

21. A device in accordance with claim 20, wherein the electronic driving componenet comprises
a microcontroller programmed to execute a sequence of operations; and
a driver unit, coupled to receive at least one output from said microcontroller unit, and coupled to drive the plurality of light emitting devices responsive to output from the microcontroller.

22. A device in accordance with claim 20, wherein the plurality of light emitting devices comprises at least one light emitting diode (LED).

23. A device in accordance with claim 21, wherein the driving component controls at least one parameter selected from a group consisting of (a) duty cycle of a drive signal coupled to at least one light emitting device of the plurality of light emitting devices, (b) repetition of an on-portion of a drive signal coupled to at least one light emitting device of the plurality of light emitting devices, and (c) relative amplitude of a drive signal coupled to at least one light emitting device of the plurality of light emitting devices.

24. A device in accordance with claim 20, further comprising a user-operable ON-OFF switch controlling delivery of operating power to the device.

25. A device in accordance with claim 20, further comprising at least one fastening strap or spring mechanism attached to the formable, flexible material.

26. A device in accordance with claim 20, wherein the device further comprises one or more legs attached to the first surface and/or the second surface.

27. A device in accordance with claim 20, wherein the formable, flexible material is substantially planar.

28. A device in accordance with claim 20, wherein the formable, flexible material is substantially rectangular.

29. A device in accordance with claim 20, wherein the device can be formed into a mask.

30. A device in accordance with claim 20, wherein the device is formed of a molded, rigid mask that substantially conforms to facial contours of a user.
